(19) United States
(12) Patent Application Publication
(10) Pub. No.: US 2024/0127451 A1
(43) Pub. Date: Apr. 18, 2024

(54) PATIENT RESPONSE-BASED BIOMARKER TOPOLOGY QUANTIFICATION AND ASSESSMENT FOR MULTIPLE TISSUE TYPES

(71) Applicant: Bristol-Myers Squibb Company, Princeton, NJ (US)

(72) Inventors: George C. Lee, North Wales, PA (US); Robin Edwards, Princeton, NJ (US); Scott Ely, Princeton, NJ (US); Daniel N. Cohen, Princeton, NJ (US); John B. Wojcik, Princeton, NJ (US); Vipul A. Baxi, Hillsborough, NJ (US); Dimple Pandya, Princeton, NJ (US); Jimena Trillo-Tinoco, Princeton, NJ (US); Benjamin J. Chen, Cambridge, MA (US); Andrew Fisher, Princeton, NJ (US); Falon Gray, Princeton, NJ (US)

(73) Assignee: Bristol-Myers Squibb Company, Princeton, NJ (US)

(21) Appl. No.: 18/547,751

(22) PCT Filed: Feb. 25, 2022
(86) PCT No.: PCT/US2022/017840

§ 371 Date: Aug. 24, 2023

Related U.S. Application Data
(63) Continuation of application No. 63/153,723, filed on Feb. 25, 2021.

(51) Int. Cl. G06T 7/00; G06T 7/73; G06V 20/69; G16H 50/30

(52) U.S. Cl. CPC G06T 7/0016; G06T 7/74; G06V 20/695; G06V 20/698; G16H 50/30; G06T 2207/30024; G06T 2207/30096; G06T 2207/30204; G06V 2201/03

(57) ABSTRACT

Described herein are methods and computer systems for classification of CD8 T-cell topology using a patient response-based linear cutoff model. A plurality of histology images of tissue samples in a plurality of patients are received by a computer system. An image analysis of the plurality of histology images is performed to obtain a CD8+ T-cell abundance in the tumor parenchyma and stroma in each of the plurality of histology images. Real inflammation scores and tumor infiltration scores are determined based on a polar coordinate transformation of the CD8+ T-cell abundance in the tumor parenchyma and stroma. Based on the real inflammation scores and tumor infiltration scores, a feature space is generated, and linear boundaries or linear cutoffs between a plurality of classifications in the feature space are identified based on the real inflammation scores, the tumor infiltration scores, and patient response data.

### CROSS-REFERENCE TO EARLIER FILED APPLICATION

This PCT application claims the priority benefit of U.S. Provisional Application No. 63/153,723 filed on Feb. 25, 2021, which is incorporated by reference herein in its entirety.

### BACKGROUND

### Field

Embodiments of the present disclosure relate to methods and devices for quantification, classification, and assessment of tissue-based biomarker topology using linear cutoffs and a patient response-based model.

### Background

Biomarkers can be used to identify and assess biological processes within the body. Biomarkers are increasingly being used to assess the likelihood of particular patient outcomes for different types of treatments, so that the right treatment (e.g., medical, pharmaceutical, etc.) may be provided to a given patient. Some biomarkers are generated as an immune system response to, for example, the presence of cancerous cells or tumors, fibrosis, gastrointestinal disorders, cardiac disease, and the like.

One such example biomarker is CD8. CD8 is a transmembrane glycoprotein that may be expressed in cytotoxic T lymphocytes. Measuring the number of CD8+ tumor-infiltrating lymphocytes (TILs) can be a reliable marker for assessing immune response to cancer and determining whether a given patient is or will be responsive to various cancer immunotherapies.

### SUMMARY

In the embodiments presented herein, methods and systems are described for utilizing inflammation and tumor infiltration scores and patient response for classification of biomarker topology in any number of clinical and commercial samples for various cancers.

In an embodiment, a computer-implemented method for classification of CD8 tumor topology is described. The method includes receiving a plurality of histology images of tumor samples in a plurality of patients, performing an image analysis of the plurality of histology images to obtain CD8+ T-cell abundance in the tumor parenchyma and stroma in each of the plurality of histology images, determining, by the at least one processor, real inflammation scores and tumor infiltration scores based on a polar coordinate transformation of the CD8+ T-cell abundance in the tumor parenchyma and stroma, generating a feature space based on the real inflammation scores and the tumor infiltration scores, and identifying linear boundaries or linear cutoffs between a plurality of classifications in the feature space based on the real inflammation scores, the tumor infiltration scores, and patient response data.

Another embodiment includes a system for classification of CD8 tumor topology. The system may include a memory and a processor coupled to the memory. In an embodiment, the processor is configured to receive a plurality of histology images of tumor samples in a plurality of patients, perform an image analysis of the plurality of histology images to obtain a CD8+ T-cell abundance in the tumor parenchyma and stroma in each of the plurality of histology images, determine real inflammation scores and tumor infiltration scores based on a polar coordinate transformation of the CD8+ T-cell abundance in the tumor parenchyma and stroma, generate a feature space based on the real inflammation scores and the tumor infiltration scores, identify linear boundaries or linear cutoffs between a plurality of classifications in the feature space based on the real inflammation scores, the tumor infiltration scores, and patient response data, and store the feature space and data regarding the linear boundaries or the linear cutoffs in the memory.

A further embodiment includes a non-transitory computer-readable medium having instructions stored thereon, execution of which, by one or more processors of a device, cause the one or more processors to perform operations. In an embodiment, the operations include receiving a plurality of histology images of tumor samples in a plurality of patients, performing an image analysis of the plurality of histology images to obtain a CD8+ T-cell abundance in the tumor parenchyma and stroma in each of the plurality of histology images, determining, by the at least one processor, real inflammation scores and tumor infiltration scores based on a polar coordinate transformation of the CD8+ T-cell abundance in the tumor parenchyma and stroma, generating a feature space based on the real inflammation scores and the tumor infiltration scores, and identifying linear boundaries or linear cutoffs between a plurality of classifications in the feature space based on the real inflammation scores, the tumor infiltration scores, and patient response data.

A further embodiment includes a computer-implemented method for classification of biomarker topology is described. The method includes receiving a plurality of histology images of tumor samples in a plurality of patients, performing an image analysis of the plurality of histology images to information about the biomarker in each of the plurality of histology images, determining real inflammation scores and tumor infiltration scores based on a polar coordinate transformation of the CD8+ T-cell abundance in the tumor parenchyma and stroma, generating a feature space based on the real inflammation scores and the tumor infiltration scores, and identifying linear boundaries or linear cutoffs between the plurality of classifications in the feature space based on the real inflammation scores, the tumor infiltration scores, and patient response data.

A further embodiment includes a method including receiving, by at least one processor of a computing device, information comprising a stromal CD8+ T-cells parameter and a parenchymal CD8+ T-cells parameter for each of a plurality of classified histology images, obtaining, by the at least one processor, a CD8+ T-cell abundance in the tumor parenchyma and stroma for each of the plurality of histology images, determining, by the at least one processor, real inflammation scores and tumor infiltration scores based on a polar coordinate transformation of the CD8+ T-cell abundance in the tumor parenchyma and stroma, generating a feature space based on the real inflammation scores and the tumor infiltration scores, and identifying, by the at least one processor, linear boundaries or linear cutoffs between a plurality of classifications in the feature space based on the real inflammation scores, the tumor infiltration scores, and patient response data.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the pertinent art to make and use the disclosure.

FIG. 1 illustrates example images of tumor tissue samples with various classifications using CD8+ histology images obtained by immunostaining, according to example embodiments.

FIG. 2 is an example diagram illustrating a methodology for image analysis and using a patient response model for tumor topology classification, according to example embodiments.

FIG. 3 is another example diagram illustrating the methodology for classification of tumor topology using image analysis and patient response-based linear cutoff approaches, according to example embodiments.

FIG. 4 is a flowchart illustrating the process for classification of CD8 tumor topology, according to example embodiments.

FIG. 5 is a flowchart illustrating the process for classifying CD8 tumor topology of a histology image using a feature space obtained by applying a patient response model and linear cutoff approaches, according to example embodiments.

FIG. 6 is a block diagram of example components of a device according to example embodiments.

Embodiments of the present disclosure will be described with reference to the accompanying drawings.

### DETAILED DESCRIPTION

The following Detailed Description refers to accompanying drawings to illustrate exemplary embodiments consistent with the disclosure. This Detailed Description will so fully reveal the general nature of the disclosure that others can, by applying knowledge of those skilled in relevant art(s), readily modify and/or adapt for various applications such exemplary embodiments, without undue experimentation, without departing from the spirit and scope of the disclosure. Therefore, such adaptations and modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings herein.

### Definitions

In order that the present disclosure can be more readily understood, certain terms are first defined. As used in this application, except as otherwise expressly provided herein, each of the following terms shall have the meaning set forth below. Additional definitions are set forth throughout the application.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

A “cancer” refers to a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

The term “biological sample” as used herein refers to biological material isolated from a subject. The biological sample can contain any biological material suitable for determining target gene expression, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one aspect, the sample is a tumor sample. In some aspects, the tumor sample can be obtained from a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

A “tumor sample,” as used herein, refers to a biological sample that comprises tumor tissue. In some aspects, a tumor sample is a tumor biopsy. In some aspects, a tumor sample comprises tumor cells and one or more non-tumor cell present in the tumor microenvironment (TME). For the purposes of the present disclosure, the TME is made up of at least two regions. The tumor “parenchyma” is a region of the TME that includes predominantly tumor cells, e.g., the part (or parts) of the TME that includes the bulk of the tumor cells. The tumor parenchyma does not necessarily consist of only tumor cells, rather other cells such as stromal cells and/or lymphocytes can also be present in the parenchyma. The “stromal” region of the TME includes the adjacent non-tumor cells. In some aspects, the tumor sample comprises all or part of the tumor parenchyma and one or more cells of the stroma. In some aspects, the tumor sample is obtained from the parenchyma. In some aspects the tumor sample is obtained from the stroma. In other aspects, the tumor sample is obtained from the parenchyma and the stroma.

In some aspects, the TME may be classified as immune desert, immune excluded, immune inflamed, or immune balanced. The term “immune desert” indicates that T-cells are minimal or absent from the TME. In some embodiments, the immune desert classification may be referred to herein as “desert” or “cold.” The term “immune excluded” indicates that T-cells have accumulated in the tumor stroma without efficient infiltration of the tumor parenchyma. In some embodiments, the immune excluded classification may be referred to herein as “stromal.” The term “immune inflamed” indicates that T-cells have infiltrated in the tumor parenchyma. In some embodiments, the immune inflamed classification may be referred to herein as “parenchymal.” The term “immune balanced” indicates an intermediate classification level between excluded and inflamed, in which there may be similar numbers of T-cells accumulated in the tumor stroma and T-cells accumulated in the tumor parenchyma.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### Exemplary Study of Patient Response-Based Classification of CD8-Topology

Described herein are methods for a patient response-based classification of CD8-topology on a plurality of tumor biopsies and resections using linear cutoffs and image analysis of CD8 slides and histology images obtained by immunostaining. Identification of CD8+ T-cell abundance and CD8-topology may be particularly useful to stratify patient outcomes in solid tumors based on spatial CD8+ cell patterns. Understanding the role of CD8-topology in different clinical settings may allow for more personalized treatment options for patients. In some embodiments, conducting such studies in a reproducible way may be challenging because manual interpretation of these complex patterns is subject to significant inter-reviewer variability. However, optimization of linear cutoffs using retrospective clinical data and patient response-based approaches may be useful for quantifying CD8-topology in a biologically-meaningful, reproducible, and scalable method. In particular, linear cutoff optimization for a feature space and patient response-based methodologies may be utilized to assess CD8-topology in any number of clinical and commercial CD8 histology slides for various cancers.

Although the examples herein are described in the context of CD8, the methods described herein may also be utilized to identify and classify the topology of other antigens and biomarkers for detecting additional tumors or cancerous cells. Biomarkers include, but are not limited to, PD-L1, PD-1, LAG3, CLTA-4, TIGIT, TIM3, NKG2a, CSF1R, OX40, ICOS, MICA, MICB, CD137, KIR, TGFβ, IL-10, IL-8, B7-H4, Fas ligand, CXCR4, mesothelin, CD27, GITR, and any combination thereof. The markers may also include morphologically identified markers without a staining antibody, such as lymphocytes, fibroblasts, macrophages, neutrophils, eosinophils, or any combination thereof. Similarly, although the examples herein are described in the context of tumors, the patient response-based methods described herein may also be applicable for other tissue types in a variety of therapeutic uses, such as in fibrosis, cardiological, gastrointestinal, and other oncologic and non-oncologic therapeutic areas.

### Exemplary Embodiments of Cutoff Optimization and Patient Response-Based Assessment of Tumor Topology

Inflammation of the tumor microenvironment (TME), marked by infiltration of CD8+ T-cells, has been associated with improved clinical outcomes across multiple tumor types. Parenchymal infiltration of CD8+ T-cells has been associated with improved survival with immuno-oncology (I-O) treatment, and intratumoral localization also affects outcome, highlighting the importance of spatial analysis of CD8+ T-cells within the TME. CD8+ T-cell patterns within tumors, as assessed by immunostaining of histology images, are variable and may be classified as: (i) immune desert (minimal T-cell infiltrate); (ii) immune excluded (T-cells confined to tumor stroma or invasive margin); or (iii) Immune inflamed (T-cells infiltrating tumor parenchyma, positioned in proximity to tumor cells). Artificial intelligence (AI)-based image analysis can be used to characterize the tumor parenchymal and stromal compartments in the TME.

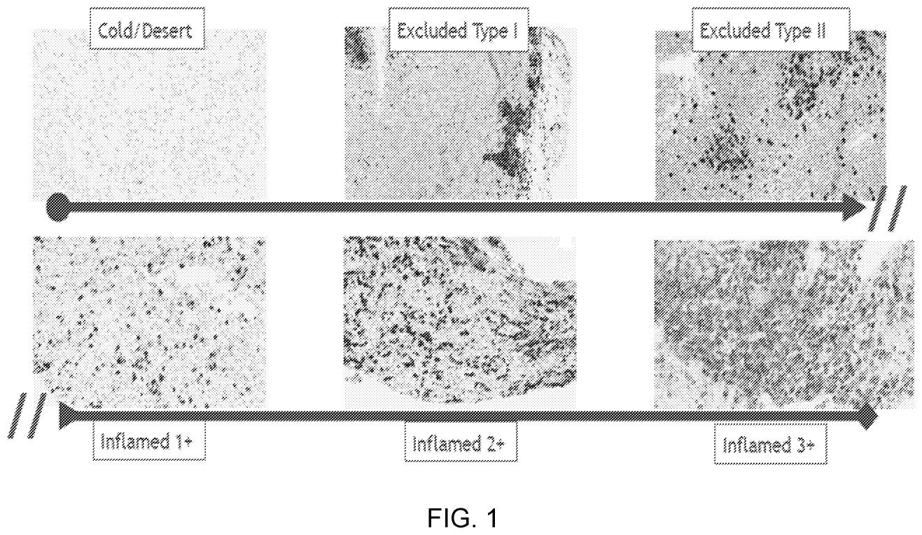

FIG. 1 illustrates example images of tumor tissue samples with various classifications using CD8+ histology images obtained by immunostaining, according to example embodiments. The tumor images show the various classifications of CD8+ T-cell patterns within the TME. The images in the top row in FIG. 1 show the immune desert and immune excluded classifications, and the images in the bottom row of FIG. 1 show the immune inflamed classification.

The immune desert classification indicates that the T-cells are minimal or absent from the TME. In some embodiments, the immune desert classification may be referred to herein as “desert” or “cold.” The immune excluded classification indicates that T-cells have accumulated in the tumor stroma without efficient infiltration of the tumor parenchyma. In some embodiments, the immune excluded classification may be referred to herein as “stromal.” The immune inflamed classification indicates that T-cells have infiltrated in the tumor parenchyma. In some embodiments, the immune inflamed classification may be referred to herein as “parenchymal.”

In some embodiments, there may be different levels within the immune excluded and immune inflamed classifications (e.g., first and second excluded levels, first, second, and third inflamed levels, and so forth) depending on the progression of the T-cells migrating within the TME. In some embodiments, a third inflamed level may indicate a higher number of T-cells infiltrating the parenchyma than the number of T-cell infiltrating the parenchyma in a first inflamed level. Although not shown in FIG. 1, there may be an intermediate classification between excluded and inflamed, referred to herein as “balanced.” The term “balanced” indicates an intermediate classification level between excluded and inflamed, in which there may be similar numbers of T-cells accumulated in the tumor stroma and T-cells accumulated in the tumor parenchyma.

In some embodiments, the tumor sample in the histology images obtained by immunostaining may be obtained by tissue biopsy and/or by resection of tumor tissue. In some embodiments, the tumor sample is a tumor tissue biopsy. In some embodiments, the tumor sample is a formalin-fixed, paraffin-embedded tumor tissue or a fresh-frozen tumor tissue. In some embodiments, the tumor sample is obtained from a stroma of the tumor. In some embodiments, the histology images obtained by immunostaining may be referred to herein as histology images.

In some embodiments, CD8 topology methods might not be standardized, resulting in inter-reviewer variability from different pathologists reviewing histology images. Interpretation of the CD8 topology from HISTOLOGY images may be confounded by various factors, such as different tumor types, limited tumor architecture due to biopsy or sampling, heterogeneity of inflammation within a tumor sample, and the like.

To address these problems in the field, embodiments described herein present a solution that provides a standardized, scalable approach using image analysis and patient response-based techniques to facilitate review and assessment of CD8 topology of tumor tissue in patients.

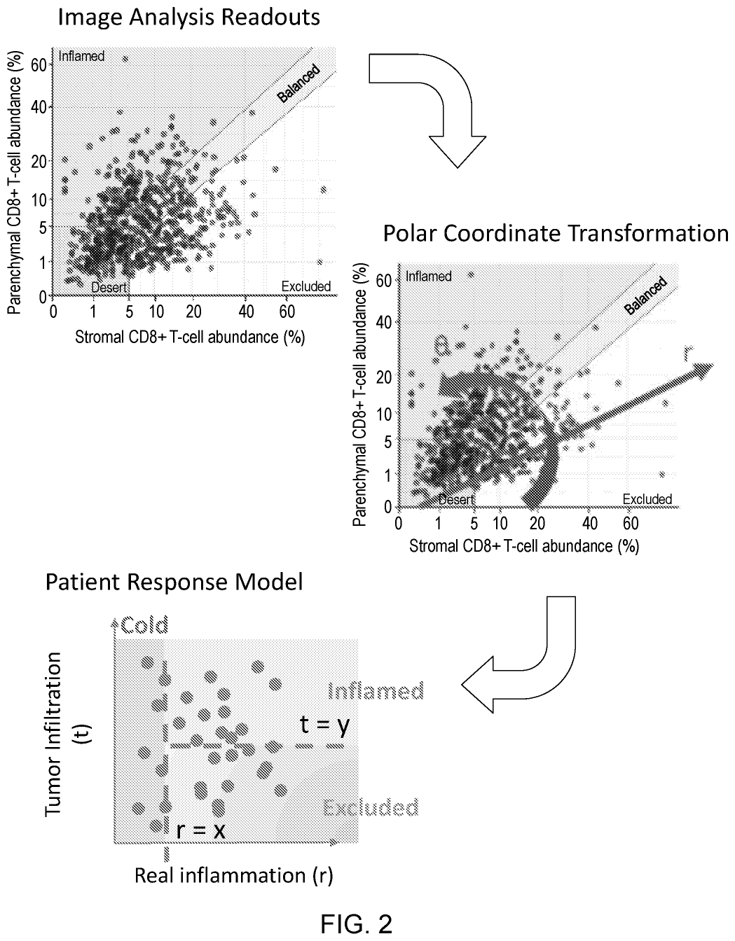

FIG. 2 is an example diagram illustrating a methodology for image analysis and patient response based approaches for tumor topology classification, according to example embodiments. In particular, FIG. 2 shows three different stages of the methodology, including image analysis, polar coordinate transformation, and applying a patient response-based model. The images used for the image analysis may include histology images obtained by immunostaining, which shows CD8+ T-cell patterns within a TME for a plurality of patients. These images may have been labelled by trained topologists as classified into various categories. In some embodiments, the classification categories are “desert,” “excluded,” and “stromal.” In some embodiments, the classification categories include “balanced.” In some embodiments, the classification category “desert” may be referred to herein as “cold,” and the classification category “stromal” may be referred to herein as “inflamed.”

In the first stage, the histology images are processed to extract information from each histology image. In some embodiments, an image analysis process identifies and outputs a variety of parameters for each image. In some embodiments, the image parameters are already known, and the image analysis process selects a subset of parameters for further analysis. Such parameters may include, for example, the number of stromal CD8+ T-cells, the number of parenchymal CD8+ T-cells, and the number of all CD8+ T-cells in each image. Other parameters may include the density of stromal CD8+ T-cells and the density of parenchymal CD8+ T-cells in each image, which may be particularly useful if the total number of all CD8+ T-cells is not known or cannot be determined.

In some embodiments, the image analysis may obtain a CD8+ T-cell abundance in the tumor parenchyma and stroma in each histology image. In some embodiments, the CD8+ T-cell abundance may be displayed via a graphical representation of a relationship between a percentage of the stromal CD8+ T-cells and a percentage of the parenchymal CD8+ T-cells with respect to the total number of T-cells present in each of the plurality of histology images, as shown by the “image analysis readout” plot of FIG. 2. In some embodiments, the graphical representation may show density, percentage, and/or quantity of stromal CD8+ T-cells and parenchymal CD8+ T-cells in each image. In some embodiments, the image analysis may comprise any image recognition, processing, and/or analysis algorithm(s). In some embodiments, the image analysis may be performed by applying an artificial neural network (e.g., a convolutional neural network) to the plurality of histology images.

In the second stage, a polar coordinate transformation may be performed on the results from the image analysis to transform the image analysis readout graph into a polar plot with polar coordinates. In some embodiments, the polar coordinate transformation may comprise a mathematical transformation of the features derived during image analysis to a polar coordinate feature space.

In the third stage, a patient response model may be applied to the transformed results of the image analysis and the CD8+ T-cell abundance in the tumor parenchyma and stroma. In some embodiments, the patient response model may include computations for determining real inflammation values (r) and tumor infiltration values (t) based on the polar coordinate transformation, and identifying linear boundaries or linear cutoffs between a plurality of classifications in a feature space based on the real inflammation scores, the tumor infiltration scores, and patient response data.

In some embodiments, real inflammation scores (r) and tumor infiltration scores (t) are defined by the polar coordinate transformation of the x-axis showing percent stromal CD8+ T-cells and the y-axis showing percent parenchymal CD8+ T-cells. In some embodiments, percent stromal CD8+ T-cells represents a number of CD8+ T-cells in the stroma divided by the total number of T-cells in the stroma, and percent parenchymal CD8+ T-cells represents a number of CD8+ T-cells in the parenchyma divided by the total number of T-cells in the parenchyma. In some embodiments, the plurality of classifications for categorizing the CD8 topology may include inflamed, desert/cold, or excluded. In some embodiments, a feature space may be generated by plotting values of the real inflammation scores and the tumor infiltration scores. In some embodiments, the plurality of classifications in the feature space may be determined by identifying linear boundaries or linear cutoffs across the derived real inflammation and tumor infiltration scores. In some embodiments, histology images may be classified as cold, excluded, or inflamed cases using the classifications in the feature space. In some embodiments, a histology image may be classified as a cold case if a value of a real inflammation score is less than a predetermined value on an X-axis of the plot of the feature space. In some embodiments, a histology image may be classified as an excluded case if a value of a real inflammation score is greater than or equal to a predetermined value on an X-axis of the plot of the feature space and value of the tumor infiltration score is less than a predetermined value on a Y-axis of the plot of the feature space. In some embodiments, a histology image may be classified as an inflamed case if a value of a real inflammation score is greater than or equal to a predetermined value on an X-axis of the plot of the feature space and if the value of the additional tumor infiltration score is greater than a predetermined value on a Y-axis of the plot of the feature space.

In addition to the real inflammation and tumor infiltration scores, patient response data may be used to identify linear boundaries or linear cutoffs between a plurality of classifications in the feature space. In some embodiments, patient response data may include clinical response data, such as retrospective clinical response data, indicating whether or not each patient in the plurality of patients is or was responsive to a particular treatment or therapy. In some embodiments, boundary-defining values for real inflammation and tumor infiltration may be iteratively fitted in the feature space until the linear boundaries or the linear cutoff between the plurality of classifications, as guided by the patient response data, are optimized.

In some embodiments, a recommendation for immunotherapy or treatment for a patient's tumor may be generated based on determining a classification for at least one histology image of the patient's tumor using the feature space obtained from the patient response model.

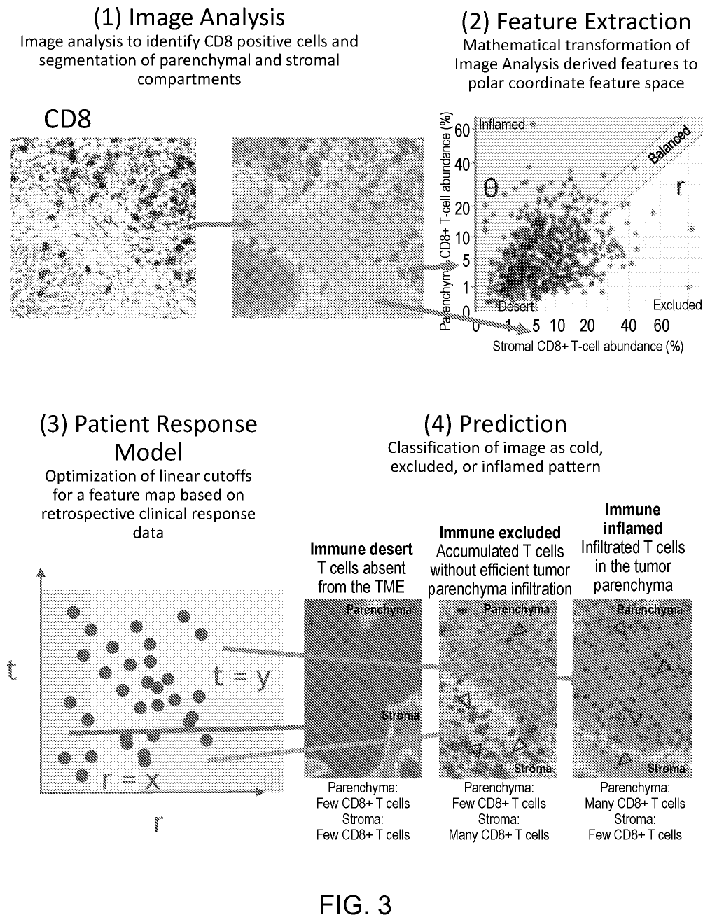

FIG. 3 is another example diagram illustrating the methodology for classification of tumor topology using image analysis and patient response-based approaches, according to example embodiments. In some embodiments, FIG. 3 illustrates additional details for an embodiment of the methodology shown in FIG. 2. FIG. 3 illustrates four stages for tumor topology classification and classifying new images using the patient response model, in which the stages include image analysis, feature extraction, patient response model, and prediction.

First, as shown in FIG. 3-(1), image analysis may be performed to identify CD8 positive cells and segmentation of parenchymal and stromal compartments in histology images of tumors. In some embodiments, the image analysis may include applying a neural network (e.g., a convolutional neural network) to a plurality of histology images to assess CD8+ T-cells in different parts of the tumor (e.g., tumor epithelium, stroma, and parenchyma) in each image. The image analysis tool may result in identifying values for a plurality of different parameters for each of the images in the plurality of histology images. In some embodiments, two parameters (e.g., number of stromal CD8+ T-cells and number of parenchymal CD8+ T-cells) may be selected for further analysis. In some embodiments, a CD8+ T-cell abundance in the tumor parenchyma and stroma for the plurality of histology images may be obtained from the image analysis.

Next, as shown in FIG. 3-(2), a feature extraction may be conducted by applying a mathematical transformation of image analysis-derived features to transform the data into a polar coordinate feature space. In some embodiments, the feature extraction may be a part of the image analysis process to identify the relationship between stromal CD8+ T-cells and parenchymal CD8+ T-cells.

After the mathematical transformation, as shown in FIG. 3-(3), a patient response model may be applied to determine linear cutoffs for a feature map for defining CD8 topology based on real inflammation scores, tumor infiltration scores, and retrospective clinical response data. In some embodiments, applying the patient response model may include generating a feature space including the plurality of classifications (e.g., inflamed, desert/cold, or excluded). In some embodiments, determining linear cutoffs or linear boundaries for the plurality of classifications may allow an adaptive identification of relevant patient populations for stratification. In some embodiments, the methods described herein may allow optimization for stratifying treatment responses.

After generating the feature space, as shown in FIG. 3-(4), the patient response model may classify the CD8 topology in new histology images as inflamed, desert, or excluded. Such a classification for a given patient's image may then be used to diagnose a patient's condition, determine an immune response of the patient, and/or be utilized to recommend or rule out treatment options for that patient.

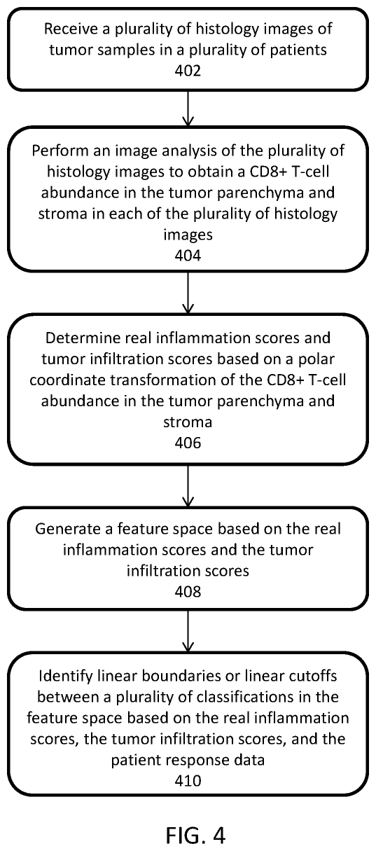

FIG. 4 is a flowchart illustrating a process for classification of CD8 tumor topology, according to example embodiments. Method 400 may be performed by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously or in a different order than shown in FIG. 4, as will be understood by a person of ordinary skill in the art.

In operation 402, a plurality of histology images of tumor samples in a plurality of patients may be received by at least one processor of a computing device. In some embodiments, the histology images may comprise tumor tissue samples obtained using CD8+ immunostaining techniques and showing CD8+ T-cell patterns within the TME for a plurality of patients.

In operation 404, an image analysis of the plurality of histology images may be performed to obtain a CD8+ T-cell abundance in the tumor parenchyma and stroma in each of the plurality of histology images. In some embodiments, performing the image analysis of the plurality of histology images includes applying an artificial neural network (e.g., a convolutional neural network) to the plurality of histology images. In some embodiments, the CD8+ T-cell abundance in the tumor parenchyma and stroma may be displayed via a graphical representation of a relationship between a percentage of the stromal CD8+ T-cells and a percentage of the parenchymal CD8+ T-cells with respect to the total number of T-cells present in each of the plurality of histology images.

In operation 406, real inflammation scores and tumor infiltration scores may be determined based on a polar coordinate transformation of the CD8+ T-cell abundance in the tumor parenchyma and stroma. In some embodiments, a polar coordinate transformation may be applied to the graphical representation of the relationship between the stromal CD8+ T-cells and parenchymal CD8+ T-cells, and the resulting polar plot may be used for determining the real inflammation scores and the tumor infiltration scores.

In operation 408, a feature space may be generated based on the real inflammation scores and the tumor infiltration scores.

In operation 410, linear boundaries or linear cutoffs between the plurality of classifications in the feature space may be identified based on the real inflammation scores, the tumor infiltration scores, and patient response data. In some embodiments, the plurality of classifications includes inflamed, desert, or excluded. In some embodiments, the feature space and data regarding the linear boundaries or the linear cutoffs between the plurality of classifications in the feature space may be stored in the memory of the computing device or computer system. In some embodiments, the patient response data includes clinical response data, such as retrospective clinical response data, indicating whether or not each patient in the plurality of patients is or was responsive to a treatment or therapy.

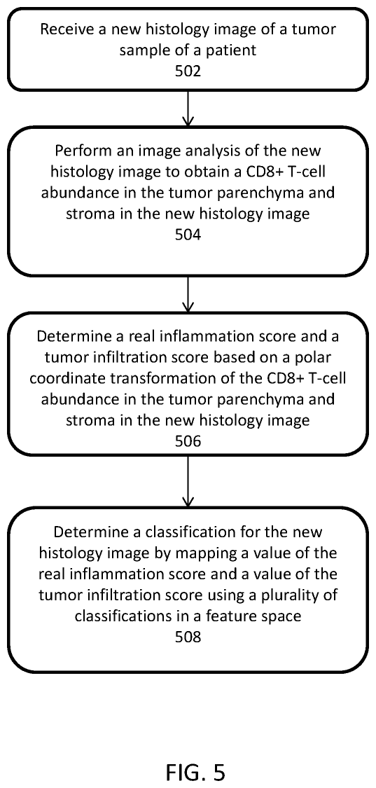

FIG. 5 is a flowchart illustrating the process for classifying CD8 tumor topology of a histology image using a feature space obtained by applying a patient response model and linear cutoff approaches, according to example embodiments. Method 500 may be performed by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously or in a different order than shown in FIG. 5, as will be understood by a person of ordinary skill in the art.

In operation 502, a new histology image of a tumor sample of a patient may be received by at least one processor of a computing device. In some embodiments, the new histology image may comprise a tumor tissue sample obtained using CD8+ immunostaining techniques and showing CD8+ T-cell patterns within the TME.

In operation 504, an image analysis of the new histology image may be performed to obtain a CD8+ T-cell abundance in the tumor parenchyma and stroma in the new histology image. This image analysis may be performed, for example, by the same image analysis algorithm(s) of operation 404 in FIG. 4.

In operation 506, a real inflammation score and a tumor infiltration score may be determined based on a polar coordinate transformation of the CD8+ T-cell abundance in the tumor parenchyma and stroma in the new histology image.

In operation 508, a classification for the new histology image may be determined by mapping a value of the real inflammation score and a value of the tumor infiltration score using a plurality of classifications in a feature space. In some embodiments, the feature space and linear boundaries or linear cutoffs between a plurality of classifications in the feature space may be generated by method 400 in FIG. 4. In some embodiments, the patient response model may be able to determine where the patterns of stromal CD8+ T-cells and parenchymal CD8+ T-cells in the new histology image fall within the boundaries for the plurality of classifications in the feature space. Based on this mapping, the patient response model may output a classification for the new histology image.

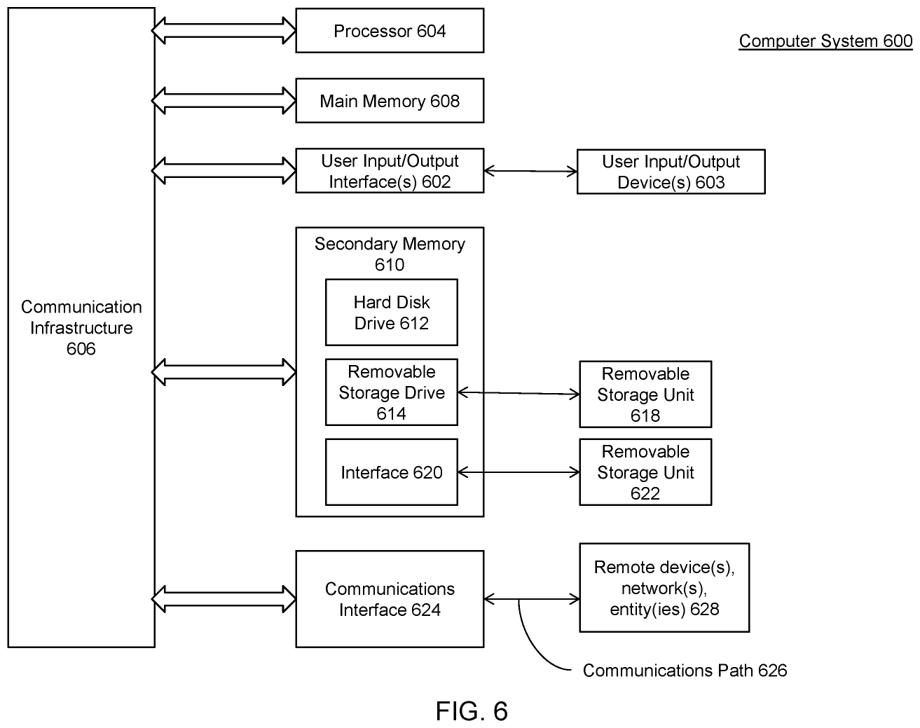

FIG. 6 is a block diagram of example components of computer system 600. One or more computer systems 600 may be used, for example, to implement any of the embodiments discussed herein, as well as combinations and sub-combinations thereof. In some embodiments, one or more computer systems 600 may be used to implement the methods 400 and 500 shown in FIGS. 4 and 5, respectively. Computer system 600 may include one or more processors (also called central processing units, or CPUs), such as a processor 604. Processor 604 may be connected to a communication infrastructure or bus 606.

Computer system 600 may also include user input/output interface(s) 602, such as monitors, keyboards, pointing devices, etc., which may communicate with communication infrastructure 606 through user input/output interface(s) 603

One or more of processors 604 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 600 may also include a main or primary memory 608, such as random access memory (RAM). Main memory 608 may include one or more levels of cache. Main memory 608 may have stored therein control logic (i.e., computer software) and/or data.

Computer system 600 may also include one or more secondary storage devices or memory 610. Secondary memory 610 may include, for example, a hard disk drive 612 and/or a removable storage drive 614.

Removable storage drive 614 may interact with a removable storage unit 618. Removable storage unit 618 may include a computer usable or readable storage device having stored thereon computer software (control logic) and/or data. Removable storage unit 618 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 614 may read from and/or write to removable storage unit 618.

Secondary memory 610 may include other means, devices, components, instrumentalities or other approaches for allowing computer programs and/or other instructions and/or data to be accessed by computer system 600. Such means, devices, components, instrumentalities or other approaches may include, for example, a removable storage unit 622 and an interface 620. Examples of the removable storage unit 622 and the interface 620 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 600 may further include a communication or network interface 624. Communication interface 624 may enable computer system 600 to communicate and interact with any combination of external devices, external networks, external entities, etc. (individually and collectively referenced by reference number 628). For example, communication interface 624 may allow computer system 600 to communicate with external or remote devices 628 over communications path 626, which may be wired and/or wireless (or a combination thereof), and which may include any combination of LANs, WANs, the Internet, etc. Control logic and/or data may be transmitted to and from computer system 600 via communication path 626.

Computer system 600 may also be any of a personal digital assistant (PDA), desktop workstation, laptop or notebook computer, netbook, tablet, smartphone, smartwatch or other wearables, appliance, part of the Internet-of-Things, and/or embedded system, to name a few non-limiting examples, or any combination thereof.

Computer system 600 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 600 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 600, main memory 608, secondary memory 610, and removable storage units 618 and 622, as well as tangible articles of manufacture embodying any combination of the foregoing. Such control logic, when executed by one or more data processing devices (such as computer system 600), may cause such data processing devices to operate as described herein.

References in the Detailed Description to “one exemplary embodiment,” “an exemplary embodiment,” “an example exemplary embodiment,” etc., indicate that the exemplary embodiment described may include a particular feature, structure, or characteristic, but every exemplary embodiment might not necessarily include the particular feature, structure, or characteristic. Moreover, such phrases are not necessarily referring to the same exemplary embodiment. Further, when a particular feature, structure, or characteristic is described in connection with an exemplary embodiment, it is within the knowledge of those skilled in the relevant art(s) to affect such feature, structure, or characteristic in connection with other exemplary embodiments whether or not explicitly described.

The exemplary embodiments described herein are provided for illustrative purposes, and are not limiting. Other exemplary embodiments are possible, and modifications may be made to the exemplary embodiments within the spirit and scope of the disclosure. Therefore, the Detailed Description is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a general purpose computer, as described above.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

The foregoing description of the specific embodiments will so fully reveal the general nature of the disclosure that others may, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

1-60. (canceled)

61. A computer-implemented method for identifying a subject suitable for immunotherapy to treat a tumor of the subject, the method executing on data processing hardware that causes the data processing hardware to perform operations comprising:
receiving a histology image of a sample of the tumor of the subject;
performing image analysis on the histology image to obtain image analysis results indicating CD8+ T-cell abundance in tumor parenchyma and stroma in the histology image;
processing a polar coordinate transformation of the image analysis results indicating the CD8+ T-cell abundance in tumor parenchyma and stroma to determine:
a real inflammation score of the sample of the tumor; and
a tumor infiltration score of the sample of the tumor;
determining, from a plurality of possible classifications of CD8 localization, a classification of CD8 localization in the sample of the tumor by applying a patient response model to the polar coordinate transformation of the image analysis results that maps a value of the real inflammation score and a value of the tumor infiltration score in a feature space, the feature space comprising a plot having a first axis and a second axis and defining linear boundaries or linear cutoffs between the plurality of possible classifications of CD8 localization; and
generating a recommendation for a treatment option of the subject based on the classification of the CD8 localization in the sample of the tumor.

62. The method of claim 61, wherein the plurality of possible classifications of CD8 localization comprises inflamed, cold, and excluded.

63. The method of claim 62, wherein determining the classification of CD8 localization in the sample of the tumor comprises determining the classification of CD8 localization in the sample of the tumor comprises cold when the mapping of the value of the real inflammation score in the feature space applied by the patient response model is less than a predetermined value on the first axis of the plot of the feature space.

64. The method of claim 62, wherein determining the classification of CD8 localization in the sample of the tumor comprises determining the classification of CD8 localization in the sample of the tumor comprises excluded when the mapping of the value of the real inflammation score is greater than or equal to a predetermined value on the first axis of the plot of the feature space and when the mapping of the value of the tumor infiltration score is less than a predetermined value on the second axis of the plot of the feature space.

65. The method of claim 62, wherein determining the classification of CD8 localization in the sample of the tumor comprises determining the classification of CD8 localization in the sample of the tumor comprises inflamed when the mapping of the value of the real inflammation score is greater than or equal to a predetermined value on the first axis of the plot of the feature space and when the tumor infiltration score is greater than a predetermined value on the second axis of the plot of the feature space.

66. The method of claim 61, wherein the operations further comprise displaying, on a screen in communication with the data processing hardware, the CD8+ T-cell abundance in the tumor parenchyma and stroma in the histology image as a graphical representation of a relationship between percentages of stromal CD8+ T-cells and percentage of parenchymal CD8+ T-cells with respect to a total number of T-cells present in the histology image.

67. The method of claim 66, wherein the operations further comprise:
determining the percentage of the stromal CD8+ T-cells by dividing a number of the CD8+ T-cells in the tumor stroma by a total number of T-cells in the tumor stroma; and
determining the percentage of the parenchymal CD8+ T-cells by dividing a number of the CD8+ T-cells in the tumor parenchyma by a total number of T-cells in the tumor parenchyma.

68. The method of claim 61, wherein the patient response model applied to the polar coordinate transformation of the image analysis results is obtained by:
receiving a plurality of histology images of tumor samples in a plurality of patients;
obtaining patient response data for each of the plurality of patients;
for each corresponding histology image in the plurality of histology images:
performing image analysis on the corresponding histology image to obtain a CD8+ T-cell abundance in a tumor parenchyma and stroma in the corresponding histology image; and
processing a polar coordinate transformation of the CD8+ T-cell abundance in the tumor parenchyma and stroma to determine:
a real inflammation score for the corresponding histology image; and
a tumor infiltration score for the corresponding histology image;
generating the feature space based on the real inflammation scores and the tumor infiltration scores determined for the plurality of histology images; and
identifying the linear boundaries or linear cutoffs between the plurality of possible classifications of CD8 localization in the feature space based on the patient response data and the real inflammation scores and the tumor infiltration scores determined for the plurality of histology images.

69. The method of claim 68, wherein the CD8+ T-cell abundance comprises a graphical representation of a relationship between percentages of stromal CD8+ T-cells and percentage of parenchymal CD8+ T-cells with respect to a total number of T-cells present in the histology image.

70. The method of claim 69, wherein the operations further comprise:
applying the polar coordinate transformation on the graphical representation, resulting in a polar plot,
wherein generating the feature space comprises using the polar plot to generate the feature space.

71. The method of claim 68, wherein the patient response data comprises retrospective clinical response data indicating whether or not each patient in the plurality of patients is responsive to a treatment or therapy.

72. The method of claim 71, wherein identifying the linear boundaries or the linear cutoff between the plurality of classifications of CD8 localization in the feature space comprises:
iteratively fitting boundary-defining values for the real inflammation scores and the tumor infiltration scores in the feature space; and
using the patient response data to identify the linear boundaries or the linear cutoff between the plurality of possible classifications of CD8 localization in the feature space.

73. A system comprising:
data processing hardware; and
memory hardware in communication with the data processing hardware and storing instructions that when executed on the data processing hardware causes the data processing hardware to perform operations for identifying a subject suitable for immunotherapy to treat a tumor of the subject, the operations comprising:
receiving a histology image of a sample of the tumor of the subject;
performing image analysis on the histology image to obtain image analysis results indicating CD8+ T-cell abundance in tumor parenchyma and stroma in the histology image;
processing a polar coordinate transformation of the image analysis results indicating the CD8+ T-cell abundance in tumor parenchyma and stroma to determine:
a real inflammation score of the sample of the tumor; and
a tumor infiltration score of the sample of the tumor;
determining, from a plurality of possible classifications of CD8 localization, a classification of CD8 localization in the sample of the tumor by applying a patient response model to the polar coordinate transformation of the image analysis results that maps a value of the real inflammation score and a value of the tumor infiltration score in a feature space, the feature space comprising a plot having a first axis and a second axis and defining linear boundaries or linear cutoffs between the plurality of possible classifications of CD8 localization; and
generating a recommendation for a treatment option of the subject based on the classification of the CD8 localization in the sample of the tumor.

74. The system of claim 73, wherein the plurality of possible classifications of CD8 localization comprises inflamed, cold, and excluded.

75. The system of claim 74, wherein determining the classification of CD8 localization in the sample of the tumor comprises determining the classification of CD8 localization in the sample of the tumor comprises cold when the mapping of the value of the real inflammation score in the feature space applied by the patient response model is less than a predetermined value on the first axis of the plot of the feature space.

76. The system of claim 74, wherein determining the classification of CD8 localization in the sample of the tumor comprises determining the classification of CD8 localization in the sample of the tumor comprises excluded when the mapping of the value of the real inflammation score is greater than or equal to a predetermined value on the first axis of the plot of the feature space and when the mapping of the value of the tumor infiltration score is less than a predetermined value on the second axis of the plot of the feature space.

77. The system of claim 74, wherein determining the classification of CD8 localization in the sample of the tumor comprises determining the classification of CD8 localization in the sample of the tumor comprises inflamed when the mapping of the value of the real inflammation score is greater than or equal to a predetermined value on the first axis of the plot of the feature space and when the tumor infiltration score is greater than a predetermined value on the second axis of the plot of the feature space.

78. The system of claim 73, wherein the operations further comprise displaying, on a screen in communication with the data processing hardware, the CD8+ T-cell abundance in the tumor parenchyma and stroma in the histology image as a graphical representation of a relationship between percentages of stromal CD8+ T-cells and percentage of parenchymal CD8+ T-cells with respect to a total number of T-cells present in the histology image.

79. The system of claim 73, wherein the operations further comprise:
determining the percentage of the stromal CD8+ T-cells by dividing a number of the CD8+ T-cells in the tumor stroma by a total number of T-cells in the tumor stroma; and
determining the percentage of the parenchymal CD8+ T-cells by dividing a number of the CD8+ T-cells in the tumor parenchyma by a total number of T-cells in the tumor parenchyma.

80. The system of claim 73, wherein the patient response model applied to the polar coordinate transformation of the image analysis results is obtained by:
receiving a plurality of histology images of tumor samples in a plurality of patients;
obtaining patient response data for each of the plurality of patients;
for each corresponding histology image in the plurality of histology images:
performing image analysis on the corresponding histology image to obtain a CD8+ T-cell abundance in a tumor parenchyma and stroma in the corresponding histology image; and
processing a polar coordinate transformation of the CD8+ T-cell abundance in the tumor parenchyma and stroma to determine:
a real inflammation score for the corresponding histology image; and
a tumor infiltration score for the corresponding histology image;
generating the feature space based on the real inflammation scores and the tumor infiltration scores determined for the plurality of histology images; and
identifying the linear boundaries or linear cutoffs between the plurality of possible classifications of CD8 localization in the feature space based on the patient response data and the real inflammation scores and the tumor infiltration scores determined for the plurality of histology images.

81. The system of claim 80, wherein the CD8+ T-cell abundance comprises a graphical representation of a relationship between percentages of stromal CD8+ T-cells and percentage of parenchymal CD8+ T-cells with respect to a total number of T-cells present in the histology image.

82. The system of claim 81, wherein the operations further comprise:
applying the polar coordinate transformation on the graphical representation, resulting in a polar plot,
wherein generating the feature space comprises using the polar plot to generate the feature space.

83. The system of claim 81, wherein the patient response data comprises retrospective clinical response data indicating whether or not each patient in the plurality of patients is responsive to a treatment or therapy.

84. The system of claim 83, wherein identifying the linear boundaries or the linear cutoff between the plurality of classifications of CD8 localization in the feature space comprises:
iteratively fitting boundary-defining values for the real inflammation scores and the tumor infiltration scores in the feature space; and
using the patient response data to identify the linear boundaries or the linear cutoff between the plurality of possible classifications of CD8 localization in the feature space.
